(19) United States
(12) Patent Application Publication
(10) Pub. No.: US 2026/0057136 A1
(43) Pub. Date: Feb. 26, 2026

(54) LATENT SPACE SIMILARITY AND LLM-BASED REDUCTIVE AND GENERATIVE OPERATIONS FOR GENERATING CHEMICAL REACTION PROTOCOLS

(71) Applicant: International Business Machines Corporation, Armonk, NY (US)

(72) Inventors: Tim ERDMANN, San Jose, CA (US); Dmitry ZUBAREV, San Jose, CA (US); Brandi RANSOM, San Jose, CA (US); Sarathkrishna SWAMINATHAN, Santa Clara, CA (US); Nathaniel H. PARK, San Jose, CA (US)

(21) Appl. No.: 18/810,759

(22) Filed: Aug. 21, 2024

(51) Int. Cl. G06F 30/20; G06F 17/16; G16C 20/10; G16C 20/70

(52) U.S. Cl. CPC G06F 30/20; G06F 17/16; G16C 20/10; G16C 20/70

(57) ABSTRACT

Method and apparatus for receiving an electronic representation of a chemical reaction, receiving a textual prompt of a problem statement regarding at least one of a first reaction protocol used for the chemical reaction; and a reaction outcome of the chemical reaction; encoding the electronic representation into a first vector using a first encoder; converting the electronic representation of the chemical reaction into a mechanistic representation of a structural change of molecules of the chemical reaction; encoding the mechanistic representation into a second vector using a second encoder; generating a vector embedding using the first vector and the second vector; querying a database using the vector embedding to retrieve a second reaction protocol; and generating a response to the textual prompt using the second reaction protocol.

### BACKGROUND

The present disclosure relates to chemical reaction protocols, and more specifically, to generating and outputting chemical reaction protocols based on user input. A chemical reaction protocol relates to the procedure for conducting a chemical reaction safely and effectively. Chemical reaction protocols can suggest the use of materials, such as reagents, solvents, equipment, or (semi-)continuous properties such as temperature, pressure, and duration, among other things. Chemical reaction protocols can also provide instructions on the preparation of reagents, such as weight and measuring certain quantities, an observed yield or conversion as often presented in reported procedures, and the setup of the reaction apparatus, which can involve using a fume hood, heating mantle, reflux condenser, etc. The protocol can include various levels of detail. For example, instructions can include the order or reagent addition, mixing techniques, or monitoring methods, among other things. The protocol can also outline steps for quenching the reaction, purifying the product, and disposing of waste. The reaction protocol can conclude with ways of characterizing the resulting product, such as nuclear magnetic resonance (NMR) or infrared (IR) among other things, as ways of confirming the reaction's success.

### SUMMARY

According to one embodiment, a method includes receiving an electronic representation of a chemical reaction; receiving a textual prompt of a problem statement regarding at least one of: a first reaction protocol used for the chemical reaction; and a reaction outcome of the chemical reaction; encoding the electronic representation into a first vector using a first encoder; converting the electronic representation of the chemical reaction into a mechanistic representation of a structural change of molecules of the chemical reaction; encoding the mechanistic representation into a second vector using a second encoder; generating a vector embedding using the first vector and the second vector; querying a database using the vector embedding to retrieve a second reaction protocol; and generating a response to the textual prompt using the second reaction protocol. Other embodiments can include a computer system or a computer-readable storage media that perform the method.

### BRIEF DESCRIPTION OF THE DRAWINGS

FIG. 1 illustrates an example operation performed by a computer system, according to some embodiments.

FIG. 2 illustrates an example flowchart of an example method performed by a computer system, according to some embodiments.

FIG. 3 illustrates an example operation performed by a computer system, according to some embodiments.

FIG. 4 illustrates an example operation performed by a computer system, according to some embodiments.

FIG. 5 illustrates an example operation performed by a computer system, according to some embodiments.

FIG. 6 illustrates an example flowchart of an example method performed by a computer system, according to some embodiments.

FIG. 7 illustrates a computing environment, according to some embodiments.

### DETAILED DESCRIPTION

According to one embodiment, a method includes receiving an electronic representation of a chemical reaction; receiving a textual prompt of a problem statement regarding at least one of: a first reaction protocol used for the chemical reaction; and a reaction outcome of the chemical reaction; encoding the electronic representation into a first vector using a first encoder; converting the electronic representation of the chemical reaction into a mechanistic representation of a structural change of molecules of the chemical reaction; encoding the mechanistic representation into a second vector using a second encoder; generating a vector embedding using the first vector and the second vector; querying a database using the vector embedding to retrieve a second reaction protocol; and generating a response to the textual prompt using the second reaction protocol. By performing the method, the response may correctly address the problem statement and may be more detailed than responses provided by existing systems.

Also in some embodiments, the method also includes receiving a natural language description of a reaction protocol; and encoding the reaction protocol into a third vector using a third encoder, where the vector embedding is generated further based on the third vector. As a result, the chemical reaction may be represented more holistically.

Also in some embodiments, the third vector of the method includes numerical values that indicate at least one of: a ratio of a reactant of the chemical reaction; or a ratio of a product of the chemical reaction. As a result, the chemical reaction may be represented more holistically.

Also in some embodiments, the method includes receiving a natural language description of a reaction outcome; and encoding the reaction outcome into a third vector using a third encoder, where the vector embedding is generated further based on the third vector. As a result, the chemical reaction may be represented more holistically.

Also in some embodiments, the third vector of the method includes numerical values that indicate at least one of a molecular structure of a reactant of the chemical reaction; or a molecular structure of product of the chemical reaction. As a result, the chemical reaction may be represented more holistically.

Also in some embodiments, the second vector of the method includes numerical values that indicate at least one of a molecular structure of a reactant of the chemical reaction; or a molecular structure of product of the chemical reaction. As a result, the chemical reaction may be represented more holistically.

Also in some embodiments, the reaction outcome of the method includes at least one of: a conversion yield of the chemical reaction; a selectivity preference of the chemical reaction; or a description of the properties and behavior of products involved in the chemical reaction. As a result, the chemical reaction may be represented more holistically.

In another embodiment, a system includes a processor set; one or more computer-readable storage media; and program instructions stored on one or more storage media to cause the processor set to perform operations including: receiving a textual prompt of a problem statement regarding at least one of: a first reaction protocol used for the chemical reaction; and a reaction outcome of the chemical reaction; encoding the electronic representation into a first vector using a first encoder; converting the electronic representation of the chemical reaction into a mechanistic representation of a structural change of molecules of the chemical reaction; encoding the mechanistic representation into a second vector using a second encoder; generating a vector embedding using the first vector and the second vector; querying a database using the vector embedding to retrieve a second reaction protocol; and generating a response to the textual prompt using the second reaction protocol. By using this system, the response may correctly address the problem statement and may be more detailed than responses provided by existing systems.

Also in some embodiments, the system also includes receiving a natural language description of a reaction protocol; and encoding the reaction protocol into a third vector using a third encoder, where the vector embedding is generated further based on the third vector. As a result, the chemical reaction may be represented more holistically.

Also in some embodiments, the third vector of the system includes numerical values that indicate at least one of: a ratio of a reactant of the chemical reaction; or a ratio of a product of the chemical reaction. As a result, the chemical reaction may be represented more holistically.

Also in some embodiments, the system includes receiving a natural language description of a reaction outcome; and encoding the reaction outcome into a third vector using a third encoder, where the vector embedding is generated further based on the third vector. As a result, the chemical reaction may be represented more holistically.

Also in some embodiments, the third vector of the system includes numerical values that indicate at least one of a molecular structure of a reactant of the chemical reaction; or a molecular structure of product of the chemical reaction. As a result, the chemical reaction may be represented more holistically.

Also in some embodiments, the second vector of the system includes numerical values that indicate at least one of a molecular structure of a reactant of the chemical reaction; or a molecular structure of product of the chemical reaction. As a result, the chemical reaction may be represented more holistically.

In another embodiment, a method includes receiving an electronic representation of a chemical reaction; receiving a mechanistic representation of a structural change of molecules of the chemical reaction; encoding the electronic representation into a first vector using a first encoder; encoding the mechanistic representation into a second vector using a second encoder; generating a vector embedding using the first vector and the second vector; and storing the vector embedding in a database. As a result, a comprehensive database regarding a chemical reaction may be created.

Also in some embodiments, the method also includes receiving a natural language description of a reaction protocol for the chemical reaction; and encoding the reaction protocol into a third vector using a third encoder, where the vector embedding is generated further based on the third vector. As a result, the chemical reaction may be represented more holistically.

Also in some embodiments, the third vector of the method includes numerical values that indicate at least one of: a ratio of a reactant of the chemical reaction; or a ratio of a product of the chemical reaction. As a result, the chemical reaction may be represented more holistically.

Also in some embodiments, the method also includes receiving a natural language description of a reaction outcome for the chemical reaction; and encoding the reaction outcome into a third vector using a third encoder, where the vector embedding is generated further based on the third vector. As a result, the chemical reaction may be represented more holistically.

Also in some embodiments, the third vector of the method includes numerical values that indicate at least one of: a reaction yield of the chemical reaction; or product properties of the chemical reaction.

Also in some embodiments, the second vector of the method includes numerical values that indicate at least one of: a molecular structure of a reactant of the chemical reaction; or a molecular structure of product of the chemical reaction. As a result, the chemical reaction may be represented more holistically.

Also in some embodiments, generating the vector embedding of the method includes appending the second vector to the first vector. As a result, the chemical reaction may be represented more holistically.

Performing the steps of a reaction protocol correctly is important for ensuring the success and reproducibility of a chemical reaction. Each step, from reagent preparation to product characterization, is designed to control variables that may affect the outcome. Missing or incorrectly performing a step, or being given an incorrect step, can lead to incomplete reactions, low yields, or the formation of unintended byproducts, among other more dangerous unwanted results. If a reaction protocol is unknown, several issues can arise, making it challenging to achieve the desired chemical transformation. Without a protocol, the precise conditions used for the reaction to take place are unclear, leading to possible inefficiencies or failure. Safety could also become a concern. Additionally, without a known protocol, reproducibility is compromised and results may not be easily validated.

Embodiments herein relate to generating complete chemical reaction protocols using an artificial intelligence (AI) system and various vector representations (embeddings) which might be stored in a vector database. To create the embeddings, different types of input data regarding a chemical reaction are received. These different inputs can each contain valuable information, but they may be presented in various formats. To ensure the elements are adequately extracted from various input types, certain encoders are used to handle certain input types. Each encoder captures the relevant information from the input it is equipped to handle and encodes the information to a vector. Together, the vectors fuse into a single vector embedding containing information from each input type regarding a chemical reaction. The vector embedding can be stored in a database containing many vector embeddings for many different chemical reactions.

The AI system can use this database. A user can input a problem statement along with general input data regarding the chemical reaction. The AI system can interpret the user input type, use an encoder equipped to handle the input type, and generate a vector containing the extracted information from user input. The vector can then be placed into a vector embedding. This generated vector embedding can be compared to the vectors existing in the database. Using techniques such as similarity analysis with or without additional metadata filtering, the appropriate information from the database can be extracted and interpreted by a large language model (LLM) within the AI system. The LLM can use the data from the database and output an appropriate response to the user's problem statement pertaining to the chemical reaction input data. The response can include a generated, complete reaction protocol used for the chemical reaction.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

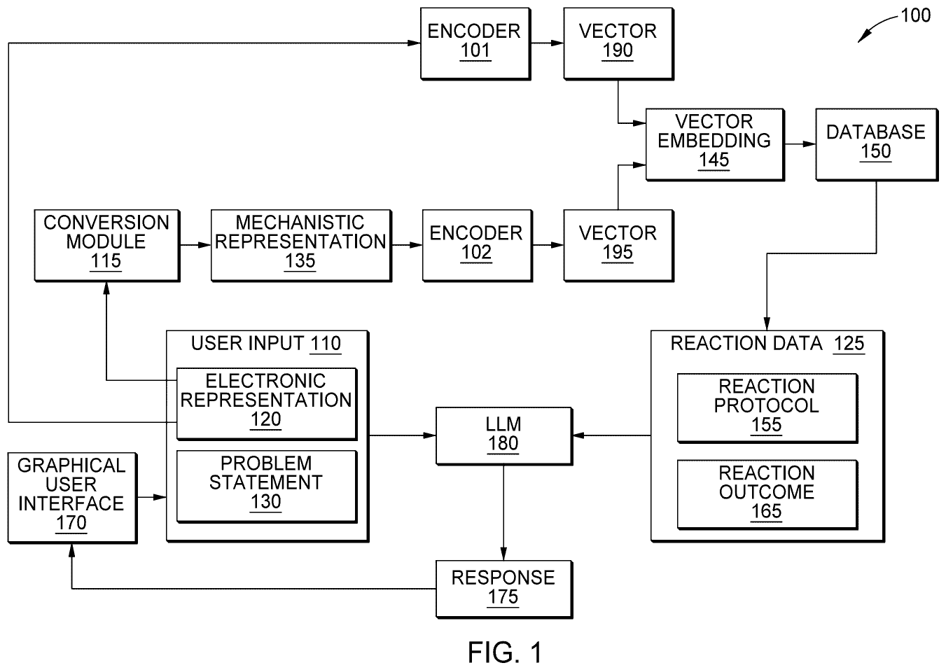

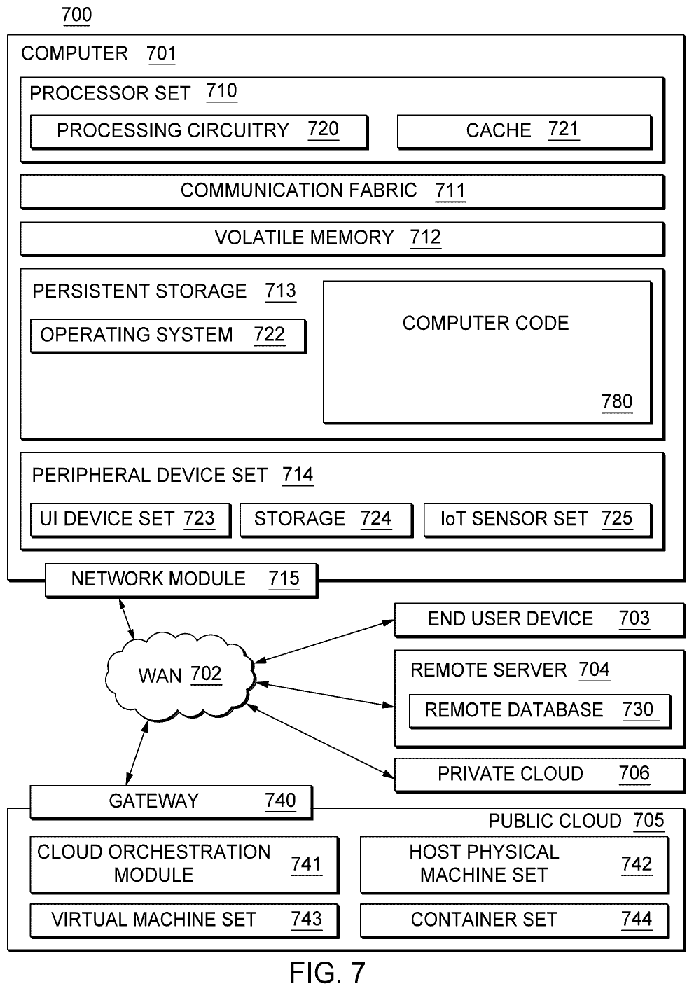

FIG. 1 illustrates an example operation 100, which may be performed by a computer system (e.g., the computer system 700 shown in FIG. 7). At a graphical user interface (GUI) 170, a user provides an input 110. Examples of the user input 110 may be an electronic representation 120 of a chemical reaction, accompanied by a problem statement 130 that the user may wish to address. The problem statement 130 may pertain to the provided electronic representation 120. The electronic representation 120 undergoes a conversion through a conversion module, 115 derive a mechanistic representation 135 of the chemical reaction from the electronic representation 120. With the electronic representation 120 and the mechanistic representation 135 both present, an encoder 101 encodes the information stored in the electronic representation 120 into a vector 190, and a separate encoder 102 encodes the information stored in the mechanistic representation 135 into a different vector 195. The vector 190 and the vector 195 are then fused to generate a vector embedding 145. The vector embedding is used to query a database 150. Latent space similarity searching may be used as the database 150 is queried using the vector embedding 145 and outputs reaction data 125. The reaction data 125 may include information from the database 150 regarding reaction protocol 155 or reaction outcome 165 as it relates to the information presented in the vector embedding 145 used to query the database 150. The reaction data 125, including the reaction protocol 155 and the reaction outcome 165, from the database 150, is presented to an LLM 180, along with the initial problem statement 130. The LLM 180 may use the reaction data 125 to generate a response 175 to the problem statement 130. The response may then be outputted to the GUI 170.

The GUI 170 may be a visual interface that allows users to interact with the system (such as computer system 700 as shown in FIG. 7) through graphical elements such as buttons, icons, menus, etc. The GUI 170 may improve the accessibility and usability of software, as it may provide more intuitive ways for users to perform actions, such as entering the user input 110, and receiving the response 175.

When interacting with the GUI 170, a user may provide user input 110. The user input 110 can include an electronic representation 120 of a chemical reaction. An electronic representation 120 of a chemical reaction refers to the digital depiction of reactants, reagents, products and the processes by which they transform. This representation may start with a chemical equation, where symbols and formulas are used to denote the reactants (or starting materials), reagents, and the products (or resulting substances). The equation may be balanced to adhere to the law of conservation of mass, showing the same number of each type of atom on both sides. Additionally, this representation can include stoichiometric coefficients that indicate the relative amounts of each substance involved in the reaction.

Beyond simple equations, the electronic representation 120 can also incorporate graphical and visual elements to provide a deeper understanding of the reaction. For example, reaction mechanisms can be depicted using arrow pushing diagrams to illustrate the step by stem movement of electros and the formation and breaking of bonds. Energy profiles can be included to show the changes in energy throughout the reaction, highlighting activation energy and the overall energy change. These detailed representations help analyze reaction pathways, predict reaction outcomes, and design new chemical processes.

One example of a particular way of electronically representing a chemical reaction can be from the simplified molecular input line entry system (SMILES). SMILES is a notation method used to represent chemical structures in a compact, text-based format. The SMILE system encodes the molecular structure of a compound using a sequence of characters, allowing for easier storage, sharing, and manipulation of chemical data in digital environments. SMILES strings convey the connectivity of atoms within a molecule, as well as information about rings, branching, and stereochemistry, using a set of predefined rules and symbols. In the context of chemical reactions, SMILES can be used to represent reactants, reagents, and products. A reaction can be described by a sequence of SMILES strings separated by two closing angle brackets, with reactants on the left, reagents in the middle, and the products on the right.

SMILES can be parsed and interpreted by computer algorithms, enabling a wide range of computational operations such as structure searching, cheminformatic calculation of properties, similarity comparisons, and molecular modeling, among other things. Additionally, SMILES strings can be converted back into graphical representations of molecules using software tools, facilitating visual inspection, and further analysis. The bidirectional convertibility between SMILES and traditional structural diagrams bridges the gap between textual and visual representations.

SMILES can also support the encoding of complex molecular features, such as chirality and isotopic labeling, among other things, which help depict the 3-dimentional arrangement of atoms and stereoisomers.

Using the electronic representation 120 of the chemical reaction, the conversion module 115 can generate a mechanistic representation 135 of the structural changes. A mechanistic representation of a structural change refers to the description of a process by which reactants transform into products at a molecular level. This representation involves illustrating the movement of electrons, the breaking and forming of chemical bonds, and the intermediate states that occur during a reaction, among other things. Arrow-pushing diagrams, known as curly arrow mechanisms, may be used to show the flow of electrons from nucleophiles (electron-rich species) to electrophiles (electron-poor species), helping visualize how reactants undergo changes to become products.

SMILES arbitrary Target Specification (SMARTS) is an extension of the SMILES notation used to define substructure patterns within molecules. While SMILES is designed to represent complete molecular structures in a compact, text-based format, SMARTS focuses on describing certain structural features or motifs that occur within a variety of chemical environments. In relation to SMILES, SMARTS builds upon the foundational syntax of SMILES, but introduces additional symbols and rules to describe sub structural elements more flexibility. For example, while a SMILES string might represent a single fully defined molecule, a SMARTS pattern can include wildcards, logical operators, and recursive definitions to match multiple molecular fragments that share common features.

SMARTS can be used to represent mechanistic aspects of reactions, such as the mechanistic representation 135 of the structural change. SMARTS patterns can capture the detailed steps of a chemical transformation. For example, a SMARTS pattern can illustrate the nucleophilic attack on an electrophilic center, the departure of a leaving group, and the formation of a new bong, among other things, which the steps represented by certain sub structural changes.

Molecular patterns, reaction classes, and molecule classes are also components of a mechanistic representations of chemical transformations. They provide a structured way of describing and analyzing the steps and features involved in chemical reactions.

Molecular patterns capture the arrangements of atoms and bonds that participate in chemical reactions. By identifying these patterns, predictions can be made regarding the way molecules will interact under certain conditions. For example, recognizing a carbonyl group in a molecule allows for predictions about its reactivity with nucleophiles. In mechanistic representations, molecular patterns help illustrate the sites of chemical activity, such as where bonds are broken and formed during a reaction. This detailed depiction aids in understand the sequence of events and the intermediate states that a molecule undergoes, providing a clearer picture of the reaction mechanism.

SMARTS can capture molecular patterns using wildcards, logical operators, and recursive definitions, among other things. Wildcards (e.g., ‘*’) can represent atoms, while other symbols may clarify atomic properties such as aromaticity (e.g. ‘a’), certain atomic numbers (e.g., ‘#6’ for carbon), or hybridization states (e.g., ‘sp2’). Logical operators allow for the combination of these properties, enabling the description of more intricate patterns. For example, the SMARTS pattern ‘[C, c]’ matches both aliphatic and aromatic carbon atoms. This flexibility makes searching for and identifying certain functional groups, among other things, easier to do.

Reaction classes categorize chemical reactions based on common mechanistic features and transformations. By grouping reactions into classes, generalizations about the behavior of different reactions that share similar mechanistic pathways can be made. Mechanistic representations use these classes to outline the possible sequences of bond making and bone breaking events, helping to predict the outcome of reactions within the same class. This classification enables systematic study of reaction mechanisms and the development of new reactions based on established patterns.

SMARTS can be used to define reaction classes by identifying certain sub structural changes that occur during a chemical transformation. This may involve writing patterns for both the reactants and the products, connected by a reaction arrow (e.g., ‘>>’). For example, the SMARTS pattern for a generic nucleophilic substitution reaction may look like “[X:1][C:2]. [Nu:1]>>[Nu:1][C:2].[X:1]” where the labels ‘:1’ and ‘:2’ track the molecule-specific atoms involved in the transformation. This pattern indicates that a nucleophile (Nu) replaces a leaving group (X) attached to a carbon atom (C).”

Molecule classes are categories of molecules that share common structural features or functional croups, influencing their chemical behavior. In mechanistic representations, identifying the class of molecules involved in a reaction helps predict their reactivity and types of transformations that can be undergone. For example, knowing that a molecule belongs to the class of alcohols implies potential reactivity with oxidizing agents or acid catalysts. This classification provides a framework for understanding how different molecules interact and transform during a reaction, supporting the prediction and rationalization of reaction mechanisms.

SMARTS can classify molecules into different classes using their structural features and functional groups. For instance, a SMARTS pattern can define the class of alcohols by indicating the presence of a hydroxyl group attached to a carbon atom, as in ‘[C][OH]’. Similarly, SMARTS can define classes of heterocycles aromatics, or other molecular groupings by including appropriate patterns that capture their characteristic substructures.

The user input 110 of an electronic representation 120 of a chemical reaction can be converted into the mechanistic representation 135 of the structural changes that occur from the chemical reaction using the conversion module 115. The conversion module 115 uses the information from the electronic representation 120 to output an accurate mechanistic representation 135 of the structural changes to the molecules involved in the chemical reaction. The process used to make this conversion may vary. One non limiting example of the processed used by the conversion module 115 includes the conversion module 115 examining the reactants and products from the electronic representation 120, which may be provided in the SMILES format. The conversion module 115 may identify functional groups and bonds in both the reactants and the products. By comparing these structures, the bonds that are broken and the bonds that are formed during the reaction may be determined. This may involve identifying the reactive sites where structural changes occur. Additionally, the conversion module 115 may map the electron flow, which shows how electrons move from one atom or bond to another during the reaction. This may be done using curved arrows to indicate the movement of electron pairs. Additionally, intermediates or transition states that occur during the reaction may be identified and depicted. These intermediates might include high energy, short lived species that form temporarily as bonds are broken and reformed. With the electron flow and intermediates mapped out, those mapped elements can be assembled into a step-by-step mechanism. The steps in the mechanism may show the starting materials, the electron movements, and the resulting intermediate structures that lead to the final products of the chemical reaction. This allows the dynamic nature of the chemical reaction to be captured, highlighting the energy changes and the structural rearrangements that occur during the chemical reaction. Additionally, the proposed mechanism may be verified and refined through comparison with experimental data and theoretical calculations. Spectroscopic evidence, kinetic studies, and computational modeling can provide insights into the feasibility and accuracy functioning of the conversion module 115. Adjustments can be made to the conversion module 115 to ensure the mechanistic representation 135 it generates aligns with observed reaction behavior and known chemical principals. This iterative functionality helps refine understanding of the reaction mechanism, leading to a more accurate and detailed description of the structural changes involved.

With both an electronic representation 120 of a chemical reaction, as well as mechanistic representation 135 of the chemical reaction depicted in the electronic representation 135, the information found in both representations is encoded into vectors. The encoder 102 encodes information from the mechanistic representation 135 of the structural changes that occur in the chemical reaction into the vector 195, and the encoder 101 encodes information from the electronic representation 120 of the chemical reaction into the vector 190.

Encoding information, which is done by both the encoder 101 and the encoder 102, refers to the process of converting data from one form to another. This transformation allows information to be represented in a standardized, compact, or more efficient format, making it easier to handle within different systems.

The encoder 102 encodes the mechanistic representation 135 of the structural changes occurring during the chemical reaction into a vector 192. The encoder may transform the detailed mechanistic pathways and interactions into a format suitable for computational analysis and modeling. The mechanistic representation 135 may describe the processes and interactions within a system, such as a biochemical pathway, or chemical reaction mechanism, and encoding this information may involve capturing the entities involved and the nature of their interactions.

Graph based representations may be used where entities (such as molecules, intermediates, enzymes, etc.) are nodes, and interactions (such as reactions, binding events, etc.) are edges. Adjacency matrices or incidence matrices can be constricted to represent these graphs. For example, an adjacency matrix may represent the presence of an interaction between two entities. To capture more detailed mechanistic information, these matrices may be augmented with additional data such as the type of interaction (e.g., catalytic, inhibitory, etc.) or kinetic parameters (e.g., rate constraints, equilibrium constraints, etc.). From these matrices, numerical vectors or feature sets can be derived, encapsulating the mechanistic details in a structured numerical form.

Another approach can involve the use of reaction fingerprints or pathway fingerprints, which encode certain features of mechanistic steps or entire pathways into binary or continuous vectors. Reaction fingerprints can capture the presence of particular reaction types, intermediates, or catalytic activities within a pathway. These fingerprints may be generated by predefined rules or machine learning models trained on known mechanistic data. For example, a vector might indicate the presence or absence of certain catalytic cycles, feedback loops, or certain molecular transformations, providing a compact and informative representation of the mechanistic process.

Furthermore, other machine learning techniques, such as graph neural networks (GNN) and recurrent neural networks (RNN), among other things, can be employed to learn continuous embeddings of mechanistic representations. GNNs can encode complex interaction networks by learning node and edge features that capture both local and global patterns within the mechanistic pathway. Similarly, RNNs can model sequential dependencies in reaction mechanisms, such as the order of intermediate steps in a multi-step reaction. By training on datasets of mechanistic pathways, these neural networks can develop sophisticated numerical vectors that encapsulate detailed mechanistic information, facilitating tasks such as mechanism prediction, pathway optimization, and the discovery of novel mechanistic insights.

The encoder 101 encodes the electronic representation 120 of the chemical reaction into a different vector 190. Encoding portions of an electronic representation of a chemical reaction into a numerical vector may involve capturing the electronic properties of the reactants, intermediates, and products, as well as the changes that occur during the reaction. This detailed information can include electron density distributions, molecular orbital energies, partial changes, and transition states, which can help understand the reaction mechanisms and kinetics.

One approach may be to use quantum chemical calculations to derive descriptors for the species involved in the reaction. Methods such as Density Functional Theory (DFT) or Hartree-Fock calculations can provide detailed electronic properties such as HOMO (highest occupied molecular orbital) and LUMO (lowest unoccupied molecular orbital) energies, electron densities, and dipole moments. These descriptors can help understand the reactivity and stability of the molecules. By calculating these properties for reactions, intermediates, and products, and capturing the changes in these properties during the reaction, a comprehensive feature vector can be constructed. This vector might include differences in HOMO-LUMO gaps, changes in electron densities, and shits in dipole moments, among other properties.

Another technique can involve using reaction fingerprints, which are extensions of molecular fingerprints but designed to capture the details of chemical reactions. Reaction fingerprints may include information about the types of bonds broken and formed, changes in atomic charges, and variations in molecular orbitals throughout the reaction process. These fingerprints can be binary vectors indicating the presence of absence of certain features or more complex vectors the quantify the extent of these changes. This method provides a compact and informative way to encode the electronic aspects of the reaction mechanism

Additionally, other machine learning models, such as GNNs and RNNs, can also be employed to encode electronic representations of chemical reactions. GNNs treat molecules as graphs and can incorporate electronic properties at the atomic bond levels, learning to generate continuous vectors that encapsulate the electronic changes during the reaction. RNNs, on the other hand, can model the sequential nature of reaction steps, capturing the progression of electronic changes from reactants to products. By training on datasets of chemical reactions, these models can learn to produce sophisticated numerical vectors that reflect detailed electronic information, which can then be used for predicting reaction outcomes, optimizing reaction conditions, and discovering new reaction pathways.

The encoding approaches used by encoder 101 and encoder 102 are not limited to those discussed above.

The vector 190 and the vector 195, containing encoded information of the electronic representation 120 of a chemical reaction as well as the mechanistic representation 135 of the structural changes occurring during that chemical reaction respectively, combine to create the vector embedding 145. Fusing together the vector 190 and the vector 195 into one vector embedding 145 involves combining the information contained in both vectors to create a single, more comprehensive, representation. This process can be achieved through various techniques such as concatenation, element wise operations, or more sophisticated such as neural network-based fusion, among other things. Concatenation involves appending one vector to the end of another, preserving the original information of both in one longer vector. Element wise operations blend the vectors' components by performing operations such as addition, which sums corresponding elements, or multiplication, which combines corresponding elements to capture interactions between features.

Advanced methods, such as using neural networks, can create more complex and informative fused embeddings. For example, multi-layer perceptrons (MLPs) or attention mechanisms can learn to weigh and combine the elements of the input vectors in a way that maximizes the usefulness of the combined representation for a certain task. These methods can capture non-linear relationships and interactions between the features of the original vectors, resulting in fused vector embeddings that encapsulate a richer set of information.

The fusing approaches use to fuse the vector 190 to the vector 195 to create the vector embedding 145 are not limited to those discussed above.

Using the vector embedding 145, the database 150 can be queried such that it outputs additional or relevant reaction data 125 for the chemical reaction encoded in the vector embedding 145.

Retrieving information from a database using a vector embedding, such as the database 150 using the vector embedding 145, can involve a process where a query vector is compared to stored vectors of the database to identify the most similar vectors. This process may be conducted in the latent space, where complex, high dimensional data is represented in a more compact and meaningful form. The similarity between vectors may be assess used various metrics, such as cosine similarity, Euclidean distance, or more sophisticated methods such as dot product similarity, depending on the nature of vectors and the application.

Cosine similarity measures the cosine of the angle between two vectors, providing a similarity score that ranges from −1 (completely dissimilar) to 1 (completely similar). In this method, vectors are normalized to unit length, and the cosine of the angle between them is computed. Euclidean distance measures the straight-line distance between two points in the latent space.

More advanced methods involve using machine learning models to project vectors into latent space while similar vectors are closer together. This can involve training models, such as auto encoders or using pre-trained embeddings. In these cases, the model learns a transformation that maps input data into latent space where semantically similar items are closer together. Once in this latent space, similarity searches can be performed efficiently, using methods like k-nearest neighbors search, among others.

When querying the database, the query vector may be transformed into the same latent space as the stored vectors. Similarity search metrics such as those discussed above may then me used to compare the query vector to the stored vectors. The vectors that are most similar to the query vector are identified as the results. This process may involve calculating the similarity score of each pair and selecting the vectors with the highest scores. In practice, this can be optimized using data structures such as KD-trees or approximate nearest neighbor search algorithms to speed up retrieval processes.

The similarity search methods described above are non-limiting in the context of this disclosure.

After the database 150 is queried using the vector embedding 145, the resulting reaction data 125 may be decoded and extracted. The decoding and extraction process involves transforming the retrieved vector embeddings, such as the vector embedding from the database 150 containing the reaction data 125, into a more readable format, such as human readable format, or extracting meaningful information from the embeddings. The extracted information, such as the reaction data 125, is decoded such that the LLM 180 can use it, alongside the user input 110, to generate a response 175 addressing the initial problem statement 130. The LLM 180 may send the response 175 back to the graphical user interface 170. This can include a predicted reaction protocol, an existing reaction protocol, a predicted reaction outcome, or known reaction outcome, among other things.

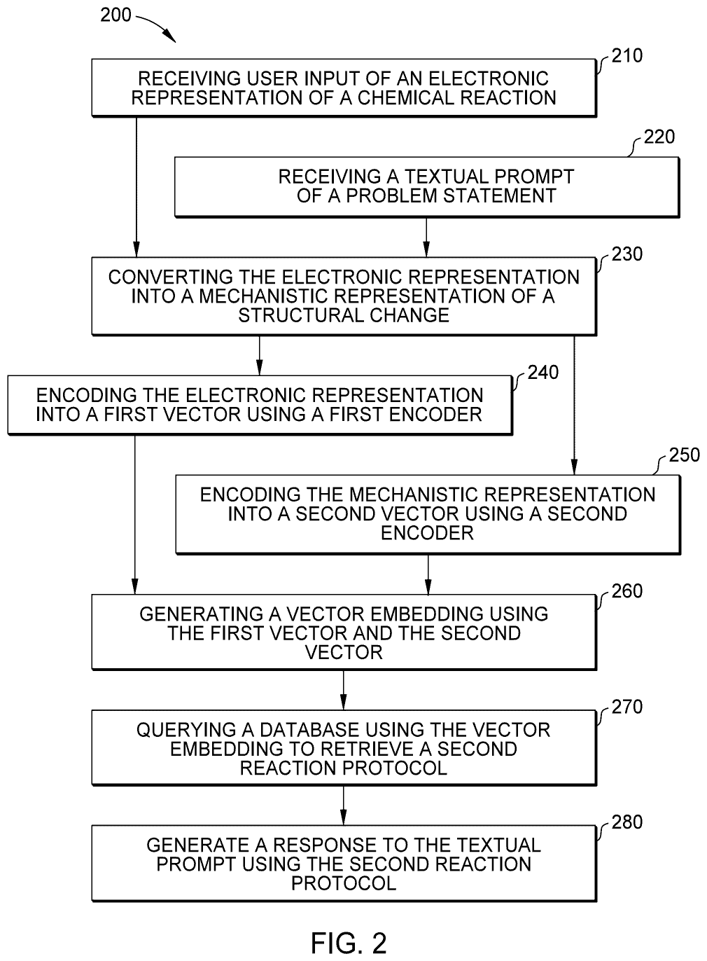

FIG. 2 is a flowchart of an example method 200, which may be performed by a computer system (e.g., the computer system 700 shown in FIG. 7). Generally, by performing the method 200, the LLM generates a response to the textual prompt, or problem statement provided by a user. This can include a predicted reaction protocol, an existing reaction protocol, a predicted reaction outcome, or known reaction outcome, among other things.

At block 210, a GUI receives user input of an electronic representation of a chemical reaction. At block 220, a GUI receives user input of a textual prompt of a problem statement. Both the electronic representation of the chemical reaction and the problem statement can be received together, and both the electronic representation of the chemical reaction and the problem statement may be related to one another. As described above, the electronic representation of the chemical reaction can be provided in many formats. Such formats include but are not limited to SMILES, international chemical identifiers (InChI), chemical markdown language (CMDL), an RXN file, or a more general notation of a chemical equation, among other things.

The problem statement can pertain to many possible areas of inquiry. For example, the elucidation of reaction mechanisms, where the process through which reactants transform into products, including the identification of intermediates and transition states, may be inquired about. Additionally, reaction kinetics may also be addressed in the problem statement. Reaction kinetics involves analyzing how different factors, such as concentration, temperature, and catalysts affect the rate of a reaction, and determining the reaction order and rate of constants. Thermodynamic analysis may also be an area of inquisition. Environmental and safety questions may also be addressed. Problem statements in this domain may focus on minimizing hazardous products, reducing energy consumption, and mitigating the environmental impact of chemical manufacturing.

The problem statement presented is not limited to the examples presented above.

At block 230, the system converts the electronic representation of the chemical reaction into a mechanistic representation of the structural changes that occur during the chemical reaction. As discussed in FIG. 1, the mechanistic representation of changes in the structure of molecules in the chemical reaction and a general electronic representation of a chemical reaction both aim to describe the chemical processes, but do so from different perspectives. Mechanistic representations focus more on the sequence of transformations and interactions that occur during a reaction. This includes detailing the formation and breaking of bonds, the formation of intermediates, and the movement of electrons through mechanisms such as nucleophilic attacks or electrophilic additions. Mechanistic diagrams may depict reaction pathways, intermediates, transition states, and electron flow, providing an improved understanding of how and why a reaction proceeds in a particular manner.

In contrast, general electronic representations may focus more on the electronic aspects of the chemical reaction. This includes but is not limited to the electron density, molecular orbital interactions, and changes in electronic states throughout the reaction. Electronic representations utilize concepts from quantum chemistry, such as molecular orbitals, electron density maps, and energy levels, to describe the reaction.

Despite the different focuses, both representations share similarities in that they aim to provide a comprehensive understanding of chemical reactions, Mechanistic representations often rely on electronic principals to explain changes in structure, whereas electronic representations sometimes use mechanistic insights to interpret chemical changes. Both approaches are complimentary, as mechanistic representations give a detailed, stepwise depiction of how molecules interact and transform, while electronic representations provide a deeper understanding of the underlying electronic forces and energy changes driving these transformations. Together, they offer a holistic view of chemical reactions, integrating both structural changes and electronic dynamics.

At block 240, an encoder in the system encodes the electronic representation of the chemical reaction into a first vector, and at block 250, a different encoder encodes the mechanistic representation of the structural changes of the molecules in the chemical reaction into a different, second vector. As described in FIG. 1, using two encoders, each suitable for handling the different input types, provides advantages to the system by tailoring the encoding process to the unique characteristics of each datatype. By focusing on the inherit properties of data, these encoders can generate more accurate and meaningful representations, For example, a specialized encoder for one datatype can capture its particular structural, semantic, or contextual feature more effectively than a general-purpose encoder. This may lead to improved performance in downstream tasks, as the embeddings produced are better aligned with the nuances of the data.

Additionally, specialized encoders may enhance computational efficiency by streamlining the encoding process for certain datatypes. Specialized encoders are designed to handle the certain complexities and structures of data, which reduces using additional processing and optimizes resource use. This targeted approach speeds up the encoding process and improves the quality of resulting representations, providing an improvement in efficiency and performance.

At block 260, the system generates a vector embedding using the first vector and the second vector. The vector embedding is generated by fusing together the two vectors that encode the mechanistic representation as well as the electronic representation. Fusing the two vectors together to generate a vector embedding, as discussed in FIG. 1, involves combining the information contained in the two vectors into a single, unified representation. The fusion may be achieved through several methods. For example, concatenation, element wise operations, or weighted averaging or neural network-based approaches. By combining multiple vectors, the resulting embedding can encapsulate a richer and more comprehensive representation of the data. This enriched embedding can enhance the performance of machine learning models by providing them with more a detailed and nuanced understanding of the input data. Additionally, vector fusion can improve data alignment and coherence, making it easier for algorithms to capture and utilize complex relationships between different data sources.

At block 270, the system queries a database using the generated vector embedding to retrieve a second reaction protocol. This can be done using similarity search techniques, as those discussed in FIG. 1. Searching a database using a vector embedding to retrieve information may involve comparing a query vector against a collection of stored vectors to identify the most similar ones. Common metrics for measuring similarity include cosine similarity, Euclidean distance, dot product similarity, etc. Once similarities are computed, the vectors with the highest similarity scores are identified. These similar vectors are then mapped back to their original entries in the database, allowing the retrieval of relevant information. For example, relevant information may be a reaction protocol for the electronic representation of the chemical reaction inputted by the user.

At block 280, an LLM generates a response to the textual prompt (problem statement) inputted by the user, and uses the information, such as the reaction protocol, retrieved from the database. The LLM interprets the contexts of the user's problem statement, along with the information retrieved from the database. By combining its understanding of the query with the retrieved data, the LLM can provide a detailed explanation, offer potential solutions, or respond in other ways as deemed appropriate.

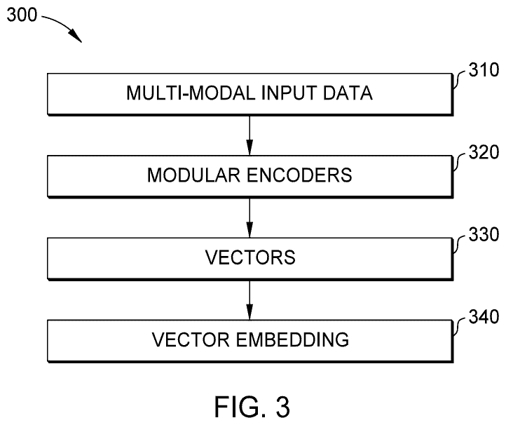

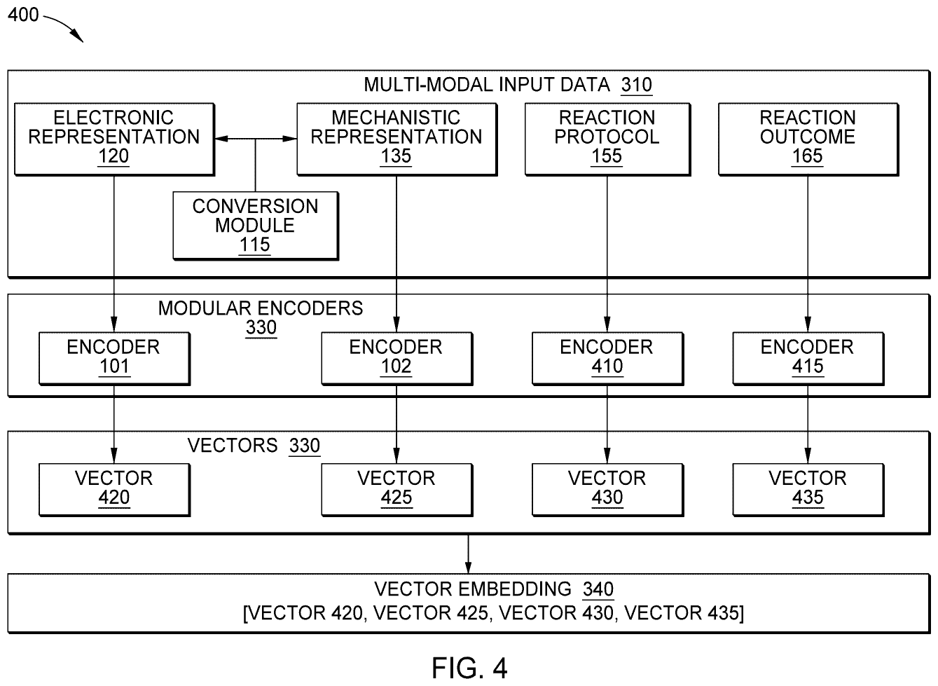

FIG. 3 illustrates an example operation 300, which may be performed by a computer system (e.g., the computer system 700 shown in FIG. 7). The operation 300 builds a vector embedding that contains relevant information about a certain chemical reaction. The vector embedding 340 is constructed using multi modal input data 310. The pieces of the multi modal input data 310 are encoded using modular encoders 320 which encode the information into separate vectors 330. The vectors 330 created are then fused into a single vector embedding 340 that contains the relevant information about the certain chemical reaction. The multi modal input data 310 may be from multiple sources and be of multiple data types. More detail is described in FIG. 4. The modular encoders 320 are multiple different encoders specialized for handling certain input types from the multi modal input data. More detail is described in FIG. 4. The modular encoders 320 produce separate vectors 330 containing information from each of the multi modal input data 310 types. The separate vectors are then fused together to create one vector embedding 340 that contains the relevant information from the multi modal input data 310 about a certain chemical reaction. More detail on this is provided in FIG. 4.

FIG. 4 illustrates an example operation 400, which may be performed by a computer system (e.g., the computer system 700 shown in FIG. 7). The multi modal input data 310 regarding a certain chemical reaction includes an electronic representation 120 of the chemical reaction, a mechanistic representation 135 of the chemical reaction, a reaction protocol 155 for the chemical reaction and a reaction outcome 165 of the chemical reaction.

As discussed in FIG. 1, the electronic representation 120 of the chemical reaction provides a detailed depiction of the way electrons are redistributed during the transformation of reactants into products. This representation may focus on the electronic structures of the molecules involved, illustrating changes in electron density, the formation and breaking of chemical bonds, and the movement of electrons through various intermediate states. It may leverage the principals of quantum chemistry and molecular orbital theory to convey the way electronic interactions drive the reaction process. Electronic representations may use various notational systems and computational models, as discussed in FIG. 1, to describe these changes.

As discussed in FIG. 1, the mechanistic representation 135 of the structural changes of molecules that occur during the chemical reaction may be derived from the electronic representation120 using the conversion module 115, or the electronic representation 120 may be derived from the mechanistic representation 135 of the structural changes.

Mechanistic representations of structural changes in molecules during a chemical reaction provide a depiction of the transformation process, illustrating the sequence of events that lead to the conversion of reactants into products. These representations may focus on the breaking and forming of chemical bonds, the generation of reaction intermediates, and the overall pathway taken by the molecules as they process through the reaction. Reaction mechanisms may be illustrated using curved arrows to indicate movement of electrons, showing the way electron pairs shift to form new bonds or break existing ones.

The relationship between the mechanistic representation 135 and the electronic representation 120 are interconnected, as both aim to describe the same chemical transformation, but from different perspectives. While mechanistic representations emphasize the structural changes and stepwise sequence of bond breaking and bond forming events, electronic representations delve into the underlying electronic phenomena driving these changes. For example, a mechanistic diagram might show a nucleophilic attack by an electron rich species on an electron deficient carbon atom, whereas the electronic representation would illustrate the way the molecular levels overlap, how electron density is redistributed, and the resulting changes in electron energy levels.

These two types of representations complement each other by providing more comprehensive understanding of chemical reactions. Mechanistic representations offer a clear and intuitive view of the reaction steps and intermediates, making it easier to visualize and predict the outcome of the reaction. Electronic representations provide deeper insight into why certain steps occur, elucidating the energetic and electronic factors that govern the reactivity and stability of molecules. Integrating mechanistic and electronic representations improves chemical research and applications.

Also included as multi modal input data 310 is the reaction protocol 155 for the chemical reaction. Reaction protocol is a detailed set of instructions that outlines the precise steps used to carry out a chemical reaction. Reaction protocol includes information on the quantities and types of reactants and solvents, the reaction conditions such as temperature, pressure, and duration, and certain techniques or equipment that may be used. Additionally, a reaction protocol can provide safety precautions, methods for monitoring the reaction process, and procedures for isolating and purifying products.

Not every type of multi modal input data 310 may be used in this system.

Reaction outcome 165 is also included as multi modal input data 310. The reaction outcome 165 refers to the results obtained after the chemical reaction is completed. The reaction outcome may encompass the identity and quality of the products formed, the yield, and the purity of these products.

The modular encoders 320 include the encoder 101, which encodes the electronic representation 120 of the chemical reaction into a vector 420, the encoder 102 which encodes the mechanistic representation 135 of the change in molecular structures of the reaction into the vector 425, the encoder 410 which encodes the reaction protocol 155 into the vector 430, and the encoder 415 which encodes the reaction outcome 165 into the vector 435. Each of the encoders handles a particular datatype and produces a numerical vector encoding valuable information pertaining to their particular datatype. As discussed in FIG. 1, specialized encoders for certain data types are designed to handle the unique characteristics of certain kinds of data, enhancing the quality and efficiency of data representation. Specialized encoders leverage domain-specific knowledge and methods to capture important features and nuances of a particular data type. The specialized encoders can in turn produce more meaningful and accurate representations.

The vectors 330 produced by the modular encoders 320, including vector 420, vector 425, vector 430, and vector 435, are then fused together into a single vector embedding 340. Fusing the vectors together into one vector embedding provides one vector that includes multi modal data about a single chemical reaction. As discussed in FIG. 1, fusing the vectors can be done in numerous ways. Fusing together multiple vectors containing different information about one topic into a single vector embedding offers numerous benefits. This action creates a comprehensive and enriched representation that captures a wide array of features and nuances from diverse data sources. The fused embedding can encapsulate various aspects of the chemical reaction, leading to more robust and informed decision-making. Moreover, a single vector embedding that integrates different pieces of information improves the efficiency and effectiveness of downstream tasks.

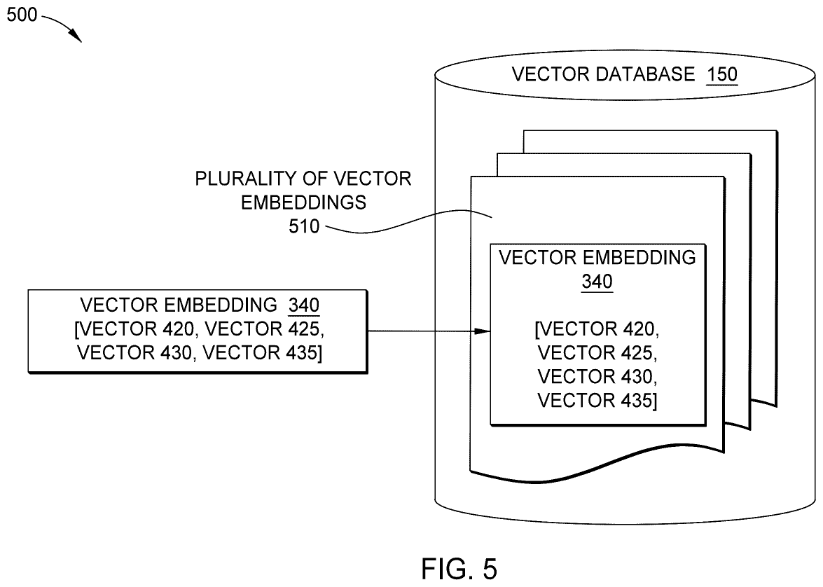

FIG. 5 illustrates an example operation 500, which may be performed by a computer system (e.g., the computer system 700 shown in FIG. 7). In the example operation 500, the vector embedding 340 is stored into the vector database 150, which is added to the collection of vector embeddings 510 already stored in the vector database 150. Storing the vector embedding 340 into the vector database 150 may involve ensuring that the data is efficiently indexed and easily retrievable for future queries. Once indexed, the vector embedding 340 may be stored along with associative metadata (identifiers, timestamps, source information, etc.) to help interpret the vector or link it other data.

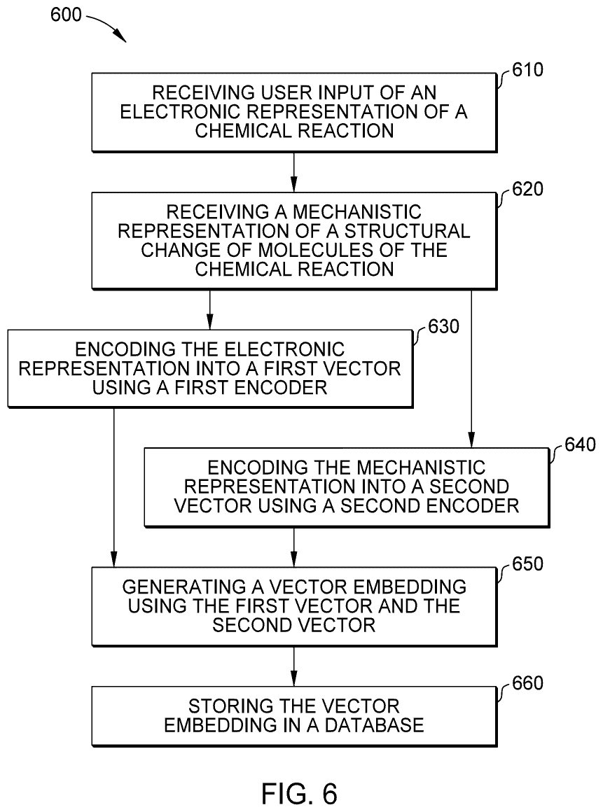

FIG. 6 is a flowchart of an example method 600, which may be performed by a computer system (e.g., the computer system 700 shown in FIG. 7). Generally, by performing the method 600, a vector embedding is stored into a database.

At block 610, the system receives user input of an electronic representation of a chemical reaction and at block 620, the system receives a mechanistic representation of the structural change of molecules of the chemical reaction. As discussed in FIG. 5, the mechanistic representation of the structural changes of a chemical reaction and the electronic representation of the chemical reaction may be derived from one another. They may also be received separately.

At block 630, the system encodes the electronic representation into a first vector, and at block 640, the system encodes the mechanistic representation into a second vector using a second encoder. As discussed in FIG. 5, the use of different encoders for different data sources offers a variety of benefits.

At block 650 the system generates a vector embedding fusing together the information of the first vector pertaining to the electronic representation of the chemical reaction, and the second vector pertaining to the mechanistic representation of the structural changes of the chemical reaction. As discussed in FIG. 5, fusing together the vectors to generate one vector embedding encapsulating information from both vectors creates a more robust representation of the chemical reaction.

At block 660, the robust vector embedding is stored in a database that may contain other vector embeddings pertaining to other, different chemical reactions. The database may be searched and used in many different applications.

In the preceding, reference is made to embodiments presented in this disclosure. However, the scope of the present disclosure is not limited to specific described embodiments. Instead, any combination of the features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the aspects, features, embodiments and advantages discussed herein are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.”

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

FIG. 7 illustrates a computing environment 700, according to some embodiments. Computing environment 700 contains an example of an environment for the execution of at least some of the computer code involved in performing the inventive methods, such as computer code 780, containing information regarding the LLM data retrieval and responses. In addition to block 780, computing environment 700 includes, for example, computer 701, wide area network (WAN) 702, end user device (EUD) 703, remote server 704, public cloud 705, and private cloud 706. In this embodiment, computer 701 includes processor set 710 (including processing circuitry 720 and cache 721), communication fabric 711, volatile memory 712, persistent storage 713 (including operating system 722 and block 780, as identified above), peripheral device set 714 (including user interface (UI) device set 723, storage 724, and Internet of Things (IoT) sensor set 725), and network module 715. Remote server 704 includes remote database 730. Public cloud 705 includes gateway 740, cloud orchestration module 741, host physical machine set 742, virtual machine set 743, and container set 744.

COMPUTER 701 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 730. As is well understood in the art of computer technology, and depending upon the technology, performance of a computer-implemented method may be distributed among multiple computers and/or between multiple locations. On the other hand, in this presentation of computing environment 700, detailed discussion is focused on a single computer, specifically computer 701, to keep the presentation as simple as possible. Computer 701 may be located in a cloud, even though it is not shown in a cloud in FIG. 1. On the other hand, computer 701 is not required to be in a cloud except to any extent as may be affirmatively indicated.

PROCESSOR SET 710 includes one, or more, computer processors of any type now known or to be developed in the future. Processing circuitry 720 may be distributed over multiple packages, for example, multiple, coordinated integrated circuit chips. Processing circuitry 720 may implement multiple processor threads and/or multiple processor cores. Cache 721 is memory that is located in the processor chip package(s) and is typically used for data or code that should be available for rapid access by the threads or cores running on processor set 710. Cache memories are typically organized into multiple levels depending upon relative proximity to the processing circuitry. Alternatively, some, or all, of the cache for the processor set may be located “off chip.” In some computing environments, processor set 710 may be designed for working with qubits and performing quantum computing.

Computer readable program instructions are typically loaded onto computer 701 to cause a series of operational steps to be performed by processor set 710 of computer 701 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 721 and the other storage media discussed below. The program instructions, and associated data, are accessed by processor set 710 to control and direct performance of the inventive methods. In computing environment 700, at least some of the instructions for performing the inventive methods may be stored in block 780 in persistent storage 713.

COMMUNICATION FABRIC 711 is the signal conduction path that allows the various components of computer 701 to communicate with each other. Typically, this fabric is made of switches and electrically conductive paths, such as the switches and electrically conductive paths that make up busses, bridges, physical input / output ports and the like. Other types of signal communication paths may be used, such as fiber optic communication paths and/or wireless communication paths.

VOLATILE MEMORY 712 is any type of volatile memory now known or to be developed in the future. Examples include dynamic type random access memory (RAM) or static type RAM. Typically, volatile memory 712 is characterized by random access, but this is not required unless affirmatively indicated. In computer 701, the volatile memory 712 is located in a single package and is internal to computer 701, but, alternatively or additionally, the volatile memory may be distributed over multiple packages and/or located externally with respect to computer 701.

PERSISTENT STORAGE 713 is any form of non-volatile storage for computers that is now known or to be developed in the future. The non-volatility of this storage means that the stored data is maintained regardless of whether power is being supplied to computer 701 and/or directly to persistent storage 713. Persistent storage 713 may be a read only memory (ROM), but typically at least a portion of the persistent storage allows writing of data, deletion of data and re-writing of data. Some familiar forms of persistent storage include magnetic disks and solid state storage devices. Operating system 722 may take several forms, such as various known proprietary operating systems or open source Portable Operating System Interface-type operating systems that employ a kernel. The code included in block 780 typically includes at least some of the computer code involved in performing the inventive methods.

PERIPHERAL DEVICE SET 714 includes the set of peripheral devices of computer 701. Data communication connections between the peripheral devices and the other components of computer 701 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 723 may include components such as a display screen, speaker, microphone, wearable devices (such as goggles and smart watches), keyboard, mouse, printer, touchpad, game controllers, and haptic devices. Storage 724 is external storage, such as an external hard drive, or insertable storage, such as an SD card. Storage 724 may be persistent and/or volatile. In some embodiments, storage 724 may take the form of a quantum computing storage device for storing data in the form of qubits. In embodiments where computer 701 is required to have a large amount of storage (for example, where computer 701 locally stores and manages a large database) then this storage may be provided by peripheral storage devices designed for storing very large amounts of data, such as a storage area network (SAN) that is shared by multiple, geographically distributed computers. IoT sensor set 725 is made up of sensors that can be used in Internet of Things applications. For example, one sensor may be a thermometer and another sensor may be a motion detector.

NETWORK MODULE 715 is the collection of computer software, hardware, and firmware that allows computer 701 to communicate with other computers through WAN 702. Network module 715 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 715 are performed on the same physical hardware device. In other embodiments (for example, embodiments that utilize software-defined networking (SDN)), the control functions and the forwarding functions of network module 715 are performed on physically separate devices, such that the control functions manage several different network hardware devices. Computer readable program instructions for performing the inventive methods can typically be downloaded to computer 701 from an external computer or external storage device through a network adapter card or network interface included in network module 715.

WAN 702 is any wide area network (for example, the internet) capable of communicating computer data over non-local distances by any technology for communicating computer data, now known or to be developed in the future. In some embodiments, the WAN 702 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 703 is any computer system that is used and controlled by an end user (for example, a customer of an enterprise that operates computer 701), and may take any of the forms discussed above in connection with computer 701. EUD 703 typically receives helpful and useful data from the operations of computer 701. For example, in a hypothetical case where computer 701 is designed to provide a recommendation to an end user, this recommendation would typically be communicated from network module 715 of computer 701 through WAN 702 to EUD 703. In this way, EUD 703 can display, or otherwise present, the recommendation to an end user. In some embodiments, EUD 703 may be a client device, such as thin client, heavy client, mainframe computer, desktop computer and so on.

REMOTE SERVER 704 is any computer system that serves at least some data and/or functionality to computer 701. Remote server 704 may be controlled and used by the same entity that operates computer 701. Remote server 704 represents the machine(s) that collect and store helpful and useful data for use by other computers, such as computer 701. For example, in a hypothetical case where computer 701 is designed and programmed to provide a recommendation based on historical data, then this historical data may be provided to computer 701 from remote database 730 of remote server 704.

PUBLIC CLOUD 705 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 705 is performed by the computer hardware and/or software of cloud orchestration module 741. The computing resources provided by public cloud 705 are typically implemented by virtual computing environments that run on various computers making up the computers of host physical machine set 742, which is the universe of physical computers in and/or available to public cloud 705. The virtual computing environments (VCEs) typically take the form of virtual machines from virtual machine set 743 and/or containers from container set 744. It is understood that these VCEs may be stored as images and may be transferred among and between the various physical machine hosts, either as images or after instantiation of the VCE. Cloud orchestration module 741 manages the transfer and storage of images, deploys new instantiations of VCEs and manages active instantiations of VCE deployments. Gateway 740 is the collection of computer software, hardware, and firmware that allows public cloud 705 to communicate through WAN 702.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 706 is similar to public cloud 705, except that the computing resources are only available for use by a single enterprise. While private cloud 706 is depicted as being in communication with WAN 702, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 705 and private cloud 706 are both part of a larger hybrid cloud.

CLOUD COMPUTING SERVICES AND/OR MICROSERVICES (not separately shown in FIG. 1): private and public clouds 106 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to as “as a service” technology paradigm where something is being presented to an internal or external customer in the form of a cloud computing service. As-a-Service offerings typically provide endpoints with which various customers interface. These endpoints are typically based on a set of APIs. One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with these things. Another category is Software as a Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

While the foregoing is directed to embodiments of the present invention, other and further embodiments of the invention may be devised without departing from the basic scope thereof, and the scope thereof is determined by the claims that follow.

What is claimed is:

1. A method comprising:
receiving an electronic representation of a chemical reaction;
receiving a textual prompt of a problem statement regarding at least one of:
a first reaction protocol used for the chemical reaction; and
a reaction outcome of the chemical reaction;
encoding the electronic representation into a first vector using a first encoder;
converting the electronic representation of the chemical reaction into a mechanistic representation of a structural change of molecules of the chemical reaction;
encoding the mechanistic representation into a second vector using a second encoder;
generating a vector embedding using the first vector and the second vector;
querying a database using the vector embedding to retrieve a second reaction protocol; and
generating a response to the textual prompt using the second reaction protocol.

2. The method of claim 1, further comprising:
receiving a natural language description of a reaction protocol; and
encoding the reaction protocol into a third vector using a third encoder, wherein the vector embedding is generated further based on the third vector.

3. The method of claim 2, wherein the third vector comprises numerical values that indicate at least one of:
a ratio of a reactant of the chemical reaction; and
a ratio of a product of the chemical reaction.

4. The method of claim 1, further comprising:
receiving a natural language description of a reaction outcome; and
encoding the reaction outcome into a third vector using a third encoder, wherein the vector embedding is generated further based on the third vector.

5. The method of claim 4, wherein the third vector comprises numerical values that indicate at least one of:
a reaction yield of the chemical reaction; or
product properties of the chemical reaction.

6. The method of claim 1, wherein the second vector comprises numerical values that indicate at least one of:
a molecular structure of a reactant of the chemical reaction; or
a molecular structure of product of the chemical reaction.

7. The method of claim 1, wherein the reaction outcome comprises at least one of:
a conversion yield of the chemical reaction;
a selectivity preference of the chemical reaction; or
a description of properties and behavior of products involved in the chemical reaction.

8. A system, comprising:
a processor set;
one or more computer-readable storage media; and
program instructions stored on one or more storage media to cause the processor set to perform operations comprising:
receiving an electronic representation of a chemical reaction;
receiving a textual prompt of a problem statement regarding at least one of:
a first reaction protocol used for the chemical reaction; and
a reaction outcome of the chemical reaction;
encoding the electronic representation into a first vector using a first encoder;
converting the electronic representation of the chemical reaction into a mechanistic representation of a structural change of molecules of the chemical reaction;
encoding the mechanistic representation into a second vector using a second encoder;
generating a vector embedding using the first vector and the second vector;
querying a database using the vector embedding to retrieve a second reaction protocol; and
generating a response to the textual prompt using the second reaction protocol.

9. The system of claim 8, wherein the operation further comprises:
receiving a natural language description of a reaction protocol; and
encoding the reaction protocol into a third vector using a third encoder, wherein the vector embedding is generated further based on the third vector.

10. The system of claim 9, wherein the third vector comprises numerical values that indicate at least one of:
a ratio of a reactant of the chemical reaction; or
a ratio of a product of the chemical reaction.

11. The system of claim 8, wherein the operation further comprises:
receiving a natural language description of a reaction outcome; and
encoding the reaction outcome into a third vector using a third encoder, wherein the vector embedding is generated further based on the third vector.

12. The system of claim 11, wherein the third vector comprises numerical values that indicate at least one of:
a reaction yield of the chemical reaction; or
product properties of the chemical reaction.

13. The system of claim 8, wherein the second vector comprises numerical values that indicate at least one of:
a molecular structure of a reactant of the chemical reaction; or
a molecular structure of product of the chemical reaction.

14. A method, comprising:
receiving an electronic representation of a chemical reaction;
receiving a mechanistic representation of a structural change of molecules of the chemical reaction;
encoding the electronic representation into a first vector using a first encoder;
encoding the mechanistic representation into a second vector using a second encoder;
generating a vector embedding using the first vector and the second vector; and
storing the vector embedding in a database.

15. The method of claim 8 further comprising:
receiving a natural language description of a reaction protocol for the chemical reaction; and
encoding the reaction protocol into a third vector using a third encoder, wherein the vector embedding is generated further based on the third vector.

16. The method of claim 9, wherein the third vector comprises numerical values that indicate at least one of:
a ratio of a reactant of the chemical reaction; or
a ratio of a product of the chemical reaction.

17. The method of claim 8, further comprising:
receiving a natural language description of a reaction outcome for the chemical reaction; and
encoding the reaction outcome into a third vector using a third encoder, wherein the vector embedding is generated further based on the third vector.

18. The method of claim 11 wherein the third vector comprises numerical values that indicate at least one of:
a reaction yield of the chemical reaction; or
product properties of the chemical reaction.

19. The method of claim 8, wherein the second vector comprises numerical values that indicate at least one of:
a molecular structure of a reactant of the chemical reaction; or
a molecular structure of product of the chemical reaction.

20. The method of claim 1, wherein generating the vector embedding comprises appending the second vector to the first vector.
